(19) United States
(12) Patent Application Publication
(10) Pub. No.: US 2008/0188498 A1
(43) Pub. Date: Aug. 7, 2008

(54) Pyridopyrimidinone compounds with antimalarial activity

(75) Inventor: Shuren Zhu, Rockville, MD (US)

Correspondence Address: Shuren Zhu;#303, 880 College Parkway, Rockville, MD, 20850, US

(73) Assignee: Radix Pharmaceuticals, Inc.

(21) Appl. No.: 11/701,660

(22) Filed: Feb. 5, 2007

(51) Int. Cl. A61K 31/519; A61K 31/505

(52) U.S. Cl. 514/264.1; 514/258.1

(57) ABSTRACT

The present invention provides pyridopyrimidinone compounds with desirable biological activity and toxicity profiles for the enhanced treatment and prevention of malaria. The invention also relates to methods of making such molecules.

### STATEMENT REGARDING FEDERALLY SPONSORED RESEARCH OR DEVELOPMENT

This invention was supported by Public Health Service Grant Number: 1R43AI063734-01 and Public Health Service Grant Number: 2R44AI063734-02 for Radix Pharmaceuticals, Inc. The U.S. Government has a paid-up license in this invention and the right in limited circumstances to require the patent owner to license others on reasonable terms as provided for by the terms of Grant Number/1R43AI063734-01 and Grant Number: 2R44AI063734-02 awarded by Public Health Service.

### CROSS-REFERENCE TO RELATED APPLICATIONS

Not Applicable.

### REFERENCE TO SEQUENCE LISTING, A TABLE, OR A COMPUTER PROGRAM LISTING COMPACT DISC APPENDIX

Not Applicable.

### FIELD OF INVENTION

The present invention relates to new compositions useful in treating malarial infections. The present invention relates to the use of pyridopyrimidinone compounds for the treatment and prevention of malaria in mammals.

### BACKGROUND OF THE INVENTION

Malaria is one of the most common infectious diseases in over 100 countries in Africa, Southeast Asia, and South America. The increasing prevalence of multiple drug resistant strains of Plasmodium falciparum in most malaria endemic areas has significantly reduced the efficacy of current anti-malarial drugs for prophylaxis and treatment of this disease. For instance, resistance to the inexpensive anti-malarial mainstays, such as chloroquine, is worldwide. Similarly, resistance to mefloquine, which was proposed as the drug of choice for chloroquine-resistant malaria, has been reported from Africa and Southeast Asia. Although drug resistance is a common problem in the treatment of most microbial infections, malaria and many neoplasms, the impact is more acute for malaria chemotherapy because of the limited number of clinically useful anti-malarial drugs. Therefore, discovering new and effective anti-malarial drugs possesses potential to contribute significantly to the economy of the nation and to the health of her people.

In 1947, Koepfli and coworkers isolated febrifugine and isofebrifugine as the active components against malaria in the Chinese herb Chang Shan (Dichroa febrifuga Lour), which has been employed by the local people as medicine against fevers caused by malaria parasites for a long time. Febrifugine acts by impairing haemazoin formation required for maturation of the parasite at the trophozoite stage. The use of febrifugine as an anti-malarial is initially appealing not only because of its rapid effect and no drug resistance, but also because of its availability. Subsequent pre-clinical researches have found that febrifugine possesses adverse side effects. Strong liver toxicity (mouse toxic death occurred at a dosage of 10 mg/kg/day) has precluded febrifugine as a clinical drug.

Structural modifications of febrifugine have been made, most of the modifications were focused on side chain alterations. It has been elucidated the essential role played by the 4-quinazolinone ring in the appearance of activity, and that the presence of a 1″-amino group and C-2′, C-3″O-functionality are crucial in the anti-malarial activity of febrifugine. Further structure-activity relationship studies demonstrated that attaching any electron-withdrawing group to the 1″-nitrogen decreases activity.

The present invention relates to new, more active and less toxic compositions for the treatment of malaria. Structural modification was made on the piperidine ring and aromatic quinazolinone moiety of febrifugine. Lower toxicity was achieved by reducing the tendency to form chemically reactive and toxic intermediates and metabolites. These compounds bear a pyrrolidine ring instead of the original piperidine ring in the parent compound febrifugine. An extra nitrogen atom was also introduced on the quinazolinone ring moiety of febrifugine.

### SUMMARY OF THE INVENTION

The present invention provides pyridopyrimidinone compounds and compositions and methods of making and using as antimalarial agents thereof. The present invention relates to improvements in the chemotherapy of malaria through the preparation of pyridopyrimidinone compounds with desirable biological activity and toxicity profiles for enhanced treatment.

Accordingly, this invention provides pyridopyrimidinone compound I: 3-[3-(3-Hydroxy-pyrrolidin-2-yl)-2-oxo-propyl]-3H-pyrido[3,2-d]pyrimidin-4-one, whose chemical structure is shown in FIG. 001.

FIG. 001

The invention also provides pyridopyrimidinone compound II: 3-[3-(3-Hydroxy-pyrrolidin-2-yl)-2-oxo-propyl]-3H-pyrido[4,3-d]pyrimidin-4-one, whose chemical structure is shown in, FIG. 002.

FIG. 002

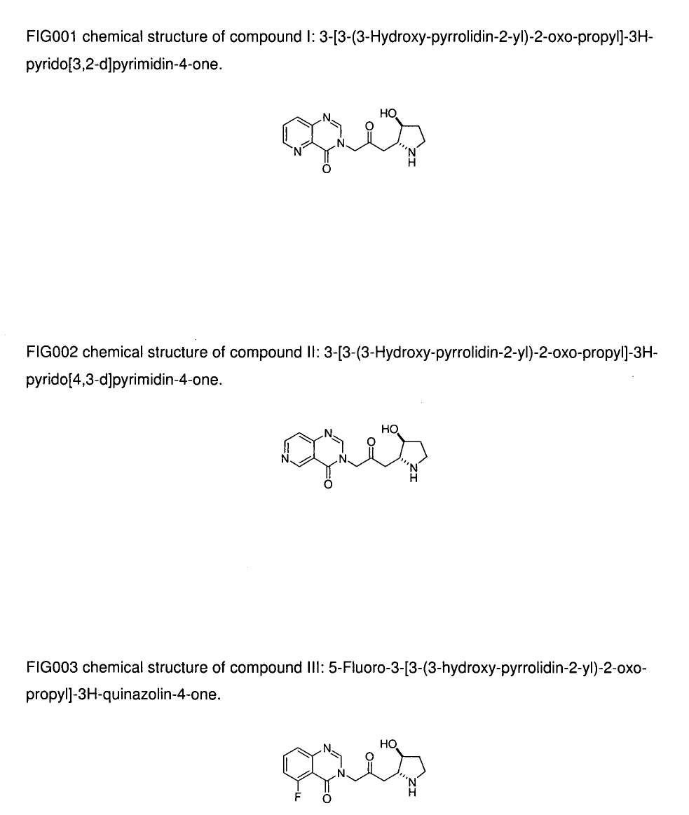

The invention also provides pyridopyrimidinone compound III: 5-Fluoro-3-[3-(3-hydroxy-pyrrolidin-2-yl)-2-oxo-propyl]-3H-quinazolin-4-one, whose chemical structure is shown in FIG. 003.

FIG. 003

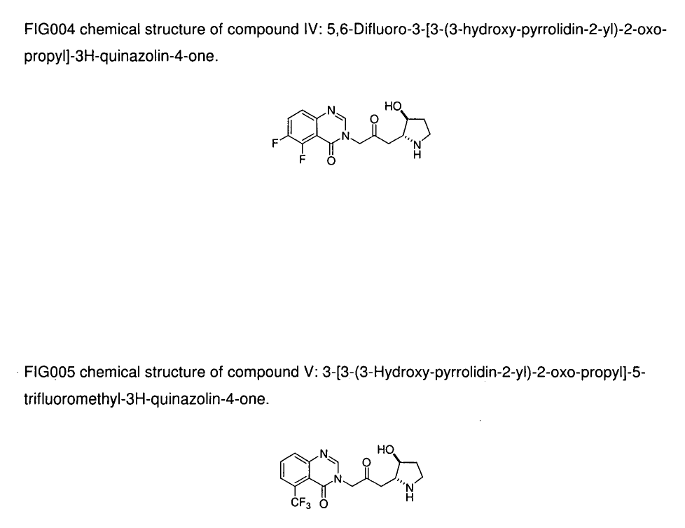

The invention also provides pyridopyrimidinone compound IV: 5,6-Difluoro-3-[3-(3-hydroxy-pyrrolidin-2-yl)-2-oxo-propyl]-3H-quinazolin-4-one, whose chemical structure is shown in FIG. 004.

FIG. 004

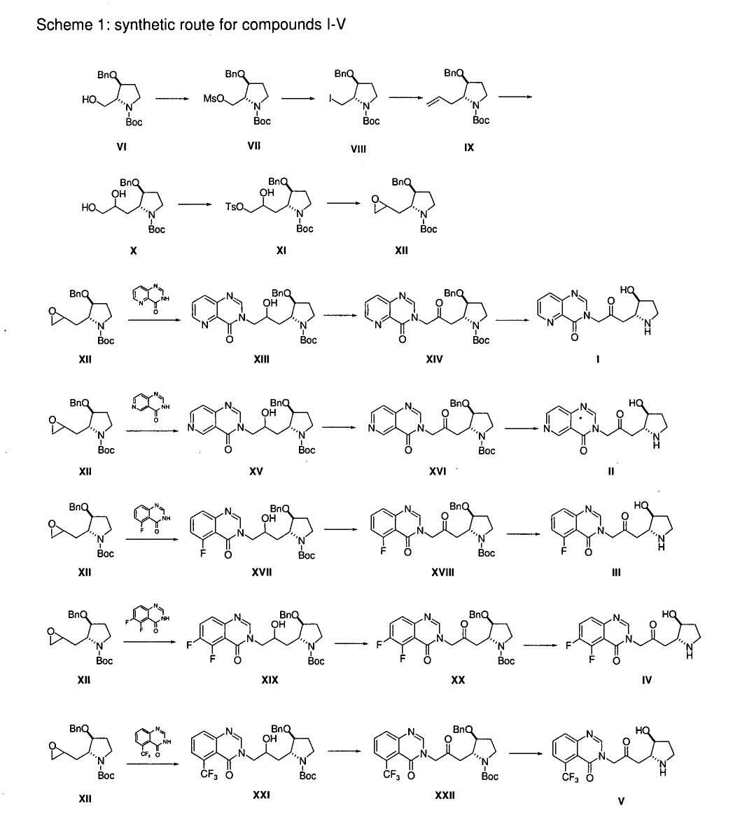

The invention also provides pyridopyrimidinone compound V: 3-[3-(3-Hydroxy-pyrrolidin-2-yl)-2-oxo-propyl]-5-trifluoromethyl-3H-quinazolin-4-one, whose chemical structure is shown in FIG. 005.

FIG. 005

Five compounds of the pyridopyrimidinone class were synthesized and their anti-parasitic activities were evaluated by administration of the test compounds to mice orally, in multiple doses. It was observed that these new compounds possessed significantly greater activity than the parent natural product febrifugine in the mouse malaria model. Hence, the present invention comprises the use of pyridopyrimidinone compounds other than febrifugine for the enhanced treatment of malarial infections. The invention also encompasses new pyridopyrimidinones which have not previously been described.

### DETAILED DESCRIPTION OF THE INVENTION

As mentioned earlier, the invention provides the following compounds:

Compound I: 3-[3-(3-Hydroxy-pyrrolidin-2-y)-2-oxo-propyl]-3H-pyrido[3,2-d]pyrimidin-4-one;

Compound II: 3-[3-(3-Hydroxy-pyrrolidin-2-yl)-2-oxo-propyl]-3H-pyrido[4,3-d]pyrimidin-4-one;

Compound III: 5-Fluoro-3-[3-(3-hydroxy-pyrrolidin-2-yl)-2-oxo-propyl]-3H-quinazolin-4-one;

Compound IV: 5,6-Difluoro-3-[3-(3-hydroxy-pyrrolidin-2-yl)-2-oxo-propyl]-3H-quinazolin-4-one;

Compound V: 3-[3-(3-Hydroxy-pyrrolidin-2-yl)-2-oxo-propyl]-5-trifluoromethyl-3H-quinazolin-4-one.

The invention is inclusive of the compounds described herein in any of their pharmaceutically acceptable forms, including isomers such as diastereomers and enantiomers, salts, solvates, polymorphs, and the like.

Preparation of the Compounds

Chemistry. Melting points were determined on a Mettler FP62 melting point apparatus and are uncorrected. Unless otherwise noted, all nonaqueous reactions were performed under an oxygen-free atmosphere of nitrogen with rigid exclusion of moisture from reagents and glassware. Analytical thin layer chromatography (TLC) was performed using EM Reagents 0.25 mm silica gel 60-F plates. Visualization of the developed chromatogram was performed by UV absorbance, aqueous potassium permanganate, or ethanolic anisaldehyde. Liquid chromatography was performed using a force flow (flash chromatography) of the indicated solvent system on EM Reagents Silica Gel 60 (70-230 mesh). Preparative TLC was performed using Whatman Silica Gel C8 TLC plates (PLK5F). Infrared spectra were recorded on a Perkin Elmer 1720 FT-IR spectrophotometer and are reported in reciprocal centimeters (cm⁻¹). ¹H and ¹³C NMR spectra were recorded in deuteriochloroform, unless otherwise noted, on a Bruker DRX-499 spectrometer at the frequency of 499 MHz. Chemical shifts are reported in parts per million on the δ scale from an internal standard of tetramethylsilane. Data are reported as follows: chemical shifts, multiplicity (s=singlet, d=doublet, t=triplet, q=quartet, qn=quintet, m=multiplet, and br=broad), coupling constant in Hertz, integration, and assignment. Combustion analyses were performed by Atlantic Microlab, Inc. (Norcross, Ga.). When necessary, solvents and reagents were dried as follows: ether, tetrahydrofuran, benzene, and toluene were stored and distilled from sodium benzophenone ketyl; dichloromethane, triethylamine, pyridine, and hexane were distilled over calcium hydride. Unless otherwise stated, the reagents were purchased from Fisher Scientific, Aldrich Chemical Company, Lancaster, or Fluka, and used as received.

The synthetic route is outlined in Scheme 1.

##Scheme 1##

3-Benzyloxy-2-methanesulfonyloxymethyl-pyrrolidine-1-carboxylic acid tert-butyl ester (VII): Mesyl chloride (MsCl, 8.0 ML) was added into a stirred solution of the known compound 3-benzyloxy-2-hydroxymethyl-pyrrolidine-1-carboxylic acid tert-butyl ester (VI) (25.3 g), triethylamine (Et₃N, 30 ML) and 4-dimethylaminopyridine (DMAP, 1.0 g) in 200 ML of methylene chloride (CH₂Cl₂) in an ice-water bath. The reaction mixture was then warmed to room temperature and stirred for additional 2 h. Solvent was evaporated. The resulting slurry was dissolved in ethyl acetate (180 ML), washed with water (150 ML), then aqueous sodium bicarbonate (80 ML), and brine (70 ML), dried over anhydrous sodium sulfate, and evaporated in a rotary evaporator under reduced pressure to furnish the essentially pure compound VII.

3-Benzyloxy-2-iodomethyl-pyrrolidine-1-carboxylic acid tert-butyl ester (VIII): Compound VII (29.0 g) and sodium iodide (NaI, 23.0 g) were dissolved in 180 ML of acetone. The resulting solution was then heated at reflux for 12 h. Solvent was evaporated. The resulting slurry was dissolved in ethyl acetate (150 ML), washed with water (130 ML), then aqueous sodium bicarbonate (60 ML), and brine (50 ML), dried over anhydrous sodium sulfate, and evaporated in a rotary evaporator under reduced pressure to furnish the crude product. Silica gel flash chromatography (15% ethyl acetate in hexanes) furnishes compound VIII as a viscous oil. Yield: 84% (2 steps). ¹H NMR: 7.37-7.26 (m, 5H), 4.50 (m, 2H), 4.09 (m, 1 H), 3.85 (m, 1 H), 3.67 (m, 1H), 3.49-3.38 (m, 3H), 2.24 (m, 1H), 1.94 (m, 1H), 1.47 (S, 9H); ¹³C NMR: 154.4, 137.9, 128.5, 127.7, 127.6, 80.2, 76.2, 70.8, 56.0, 52.8, 39.1, 28.5, 13.5.

2-Allyl-3-benzyloxy-pyrrolidine-1-carboxylic acid tert-butyl ester (IX): Vinylmagnesium bromide [1.0M in tetrahydrofuran (THF), 53.0 ML] was added into a stirred solution of compound VIII (11.0 g) and copper iodide (CuI, 2.5 g) in 100 ML of THF at −78° C. under nitrogen atmosphere. The reaction mixture was kept at −78° C. for 1 h, the slowly warmed to room temperature over a period of 2 h. The reaction mixture was then quenched with aqueous sodium bicarbonate (60 ML). After partition between ethyl acetate (120 ML) and water (120 ML), the separated organic layer was washed with brine (55 ML), dried over anhydrous sodium sulfate, and evaporated in a rotary evaporator under reduced pressure to furnish the crude product. Silica gel flash chromatography (15% ethyl acetate in hexanes) furnished compound IX as a viscous oil. Yield: 98%. ¹H NMR: 7.36-7.28 (m, 5H), 5.73 (m, 1H), 5.07 (m, 2H), 4.49 (m, 2H), 4.08 (m, 1H), 3.76 (m, 1H), 3.56 (m, 1H), 3.37 (m, 1H), 2.29-2.19 (m, 3H), 1.87 (m, 1H), 1.47 (s, 9H); ¹³C NMR: 154.8, 138.3, 134.4, 129.5, 128.5, 127.8, 117.7, 79.4, 71.3, 55.5, 51.7, 43.2, 39.1, 36.4, 28.4.

3-Benzyloxy-2-(2,3-dihydroxy-propyl)-pyrrolidine-1-carboxylic acid tert-butyl ester (X): Compound IX (9.0 g), osmium tetraoxide (OsO₄, 4% in water, 1.0 ML), and N-methyl morpholine N-oxide (NMO, 10.0 g) were dissolved in 80 ML of water and 80 ML of tetrahydrofuran (THF). The resulting heterogeneous solution is stirred vigorously for 4 days at room temperature. After partition between ethyl acetate (150 ML) and water (100 ML), the separated organic layer was washed with brine (70 ML), dried over anhydrous sodium sulfate, and evaporated in a rotary evaporator under reduced pressure to furnish the essentially pure compound X.

3-Benzyloxy-2-[2-hydroxy-3-(toluene-4-sulfonyloxy)-propyl]-pyrrolidine-1-carboxylic acid tert-butyl ester (XI): Tosyl chloride (TsCl, 5.7 g) was added into a stirred solution of compound X (8.8 g), triethylamine (Et₃N, 7.0 ML) and 4-dimethylaminopyridine (DMAP, 300 mg) in 100 ML of methylene chloride (CH₂Cl₂) in an ice-water bath. The reaction mixture was then warmed to room temperature and stirred for additional 2 h. Solvent was evaporated. The resulting slurry was dissolved in ethyl acetate (100 ML), washed with water (80 ML), then aqueous sodium bicarbonate (80 ML), and brine (50 ML), dried over anhydrous sodium sulfate, and evaporated in a rotary evaporator under reduced pressure to furnish the essentially pure compound XI.

3-Benzyloxy-2-oxiranylmethyl-pyrrolidine-1-carboxylic acid tert-butyl ester (XII): Potassium carbonate (K₂CO₃, 1.0 g) was added into a stirred solution of compound XI (11.4 g) in 25 ML of methanol (MeOH) at room temperature. After 4 h, reaction mixture was partitioned between ethyl acetate (80 ML) and water (80 ML), the separated organic layer was then washed with brine (40 ML), dried over anhydrous sodium sulfate, and evaporated in a rotary evaporator under reduced pressure to furnish the crude product. Silica gel flash chromatography (25% ethyl acetate in hexanes) furnishes compound XII as a viscous oil. Yield: 74% (3 steps). ¹H NMR: 7.35-7.28 (m, 5H), 4.88 (m, 1H), 4.58 (m, 2H), 3.74 (m, 1H), 3.39 (m, 2H), 3.05 (m, 1H), 2.79 (m, 1H), 2.52 (m, 1H), 1.87-1.82 (m, 2H), 1.46 (m, 2H), 1.44 (s, 9H); ¹³C NMR: 156.1, 138.1, 128.5, 127.8, 127.7, 79.3, 76.6, 76.0, 71.8, 49.4, 47.5, 43.3, 35.8, 34.8, 28.4.

3-Benzyloxy-2-[2-hydroxy-3-(4-oxo-4H-pyrido[3,2-d]pyrimidin-3-yl)-propyl]-pyrrolidine-1-carboxylic acid tert-butyl ester (XIII): Potassium hydride (KH, 30% in mineral oil, 3.6 g) was suspended in 50 ML of dimethylformamide (DMF). It was cooled in an ice-water bath, and solid 3H-Pyrido[3,2-d]pyrimidin-4-one (4.0 g) was added in. After 30 min, a solution of compound XII (2.2 g) in 10 ML of DMF was added in. The reaction mixture was then heated at 80° C. for 12 h under nitrogen atmosphere. It was partitioned between ethyl acetate (80 ML) and water (80 ML), separated organic layer was washed with water (3×60 ML), then brine (40 ML), dried over anhydrous sodium sulfate, and evaporated in a rotary evaporator under reduced pressure to furnish the crude product. Silica gel flash chromatography (80% ethyl acetate in hexanes) furnished compound XIII as white solid. MP: 247C. Yield: 71%. ¹H NMR: 9.11 (d, J=7.4 Hz, 1H), 8.48 (d, J=7.4 Hz, 1H), 8.24 (s, 1H), 7.85 (t, J=7.4 Hz, 1H), 7.34-7.29 (m, 5H), 5.25 (br, 1H), 4.49 (s, 2H), 4.39-4.18 (m, 3H), 3.87 (m, 1H), 3.66-3.54 (m, 2H), 3.38 (m, 1H), 2.21 (m, 1H), 1.89 (m, 1H), 1.52 (m, 2H), 1.44 (s, 9H); ¹³C NMR: 163.4, 157.0, 153.1, 149.8, 148.1, 143.8, 139.1, 132.5, 127.4, 126.9, 126.6, 122.0, 80.7, 76.6, 71.4, 71.1, 66.3, 52.4, 51.2, 42.2, 37.9, 28.4.

3-Benzyloxy-2-[2-oxo-3-(4-oxo-4H-pyrido[3,2-d]pyrimidin-3-yl)-propyl]-pyrrolidine-1-carboxylic acid tert-butyl ester (XIV): A solution of compound XIII (2.1 g) in 5 ML of CH₂Cl₂ was added into a stirred slurry of tetrapropylammonium perruthenate (TPAP, 70 mg), N-methylmorpholine N-oxide (NMO, 936 mg), and grounded molecular sieve (800 mg) in 10 ML of CH₂Cl₂ at room temperature. After 1 h, the reaction mixture was loaded directly into a short column of silica gel and eluted with 5% MeOH/EtOAc. Concentration of the eluant in a rotary evaporator under reduced pressure furnished compound XIV as white solid. MP: 229° C. Yield: 86%. [α]²⁵_{D} +17.4 (c 0.55, EtOH). ¹H NMR: 9.10 (d, J=7.3 Hz, 1H), 8.50 (d, J=7.3 Hz, 1H), 8.21 (s, 1H), 7.86 (t, J=7.3 Hz, 1H), 7.35-7.28 (m, 5H), 5.05 (d, J=17.5 Hz, 1H), 4.84 (d, J=17.5 Hz, 1H), 4.50 (m, 2H), 4.38 (m, 1H), 4.09 (m, 1H), 3.47-3.42 (m, 2H), 2.91 (m, 1H), 2.75 (dd, J=15.4 and 5.9 Hz, 1H), 2.38 (m, 1H), 1.86 (m, 1H), 1.45 (s, 9H); ¹³C NMR: 200.4, 165.1, 159.6, 155.1,150.8, 149.3, 146.7, 138.9, 133.1, 127.2, 126.9, 126.7, 121.9, 80.1, 76.6, 70.9, 53.9, 53.0, 52.2, 45.6, 37.9, 28.5.

3-[3-(3-Hydroxy-pyrrolidin-2-yl)-2-oxo-propyl]-3H-pyrido[3,2-d]pyrimidin-4-one (I): Compound XIV (1.88 g) was dissolved in 15 ML of 50% THF/H₂O. 5 ML of 6N HCl was added in. The resulting solution was heated at 80° C. for 5 hours. Solvent was evaporated. Recrystallization from ethanol-water (with addition of dilute aqueous HCl solution, 4-5 equiv. of HCl) furnishes compound I (di-HCl salt) as pale-yellow crystals. Yield: 81%. MP: 261° C. [α]²⁵_{D} +21.8 (c 0.5, EtOH). ¹H NMR: 9.72 (d, J=7.2 Hz, 1H), 9.23 (d, J=7.2 Hz, 1H), 8.81 (s, 1H), 8.22 (t, J=7.2 Hz, 1H), 5.09 (d, J=17.3 Hz, 1H), 4.91 (d, J=17.3 Hz, 1H), 4.41 (m, 1H), 4.14 (m, 1H), 3.49-3.42 (m, 2H), 2.89 (m, 1H), 2.81 (dd, J=15.1 and 6.1 Hz, 1H), 2.24 (m, 1H), 1.90 (m, 1H); ¹³C NMR: 202.1, 163.6, 159.3, 151.5, 149.6, 146.4, 132.9, 131.2, 81.0, 57.9, 54.1, 51.9, 44.7, 36.7.

3-Benzyloxy-2-[2-hydroxy-3-(4-oxo-4H-pyrido[4,3-d]pyrimidin-3-yl)-propyl]-pyrrolidine-1-carboxylic acid tert-butyl ester (XV): Synthetic procedures follow the protocols of compound XIII. Compound XV is white solid. MP: 221° C. Yield: 79%. ¹H NMR: 9.04 (s, 1H), 8.68 (d, J=7.3 Hz, 1H), 8.22 (s, 1H), 7.84 (d, J=7.3 Hz, 1H), 7.33-7.27 (m, 5H), 5.27 (br, 1H), 4.51 (s, 2H), 4.34-4.19 (m, 3H), 3.85 (m, 1H), 3.67-3.55 (m, 2H), 3.40 (m, 1H), 2.23 (m, 1H), 1.87 (m, 1H), 1.56 (m, 2H), 1.44 (s, 9H); ¹³C NMR: 167.5, 163.6, 158.0, 154.8, 148.8, 143.8, 135.1, 132.5, 129.4, 126.9, 126.6, 123.0, 80.9, 76.1, 71.9, 71.0, 65.3, 53.6, 51.6, 42.4, 37.7, 28.7.

3-Benzyloxy-2-[2-oxo-3-(4-oxo-4H-pyrido[4,3-d]pyrimidin-3-yl)-propyl]-pyrrolidine-1-carboxylic acid tert-butyl ester (XVI): Synthetic procedures follow the protocols of compound XIV. Compound XVI is white solid. MP: 221° C. Yield: 83%. [α]²⁵_{D} −24.1 (c 0.45, EtOH). ¹H NMR: 9.21 (s, 1H), 8.79 (d, J=7.4 Hz, 1H), 8.29 (s, 1H), 7.81 (d, J=7.4 Hz, 1H), 7.35-7.29 (m, 5H), 5.11 (d, J=17.4 Hz, 1H), 4.89 (d, J=17.4 Hz, 1H), 4.53 (m, 2H), 4.35 (m, 1H), 4.12 (m, 1H), 3.45-3.40 (m, 2H), 2.90 (m, 1H), 2.77 (dd, J=15.3 and 5.7 Hz, 1H), 2.37 (m, 1H), 1.88 (m, 1H), 1.47 (s, 9H); ¹³C NMR: 202.4, 166.1, 163.5, 159.7, 156.1, 149.6, 147.7, 138.7, 134.1, 128.2, 127.9, 126.8, 122.9, 81.1, 76.5, 70.4, 54.6, 53.5, 53.2, 45.4, 38.0, 28.5.

3-[3-(3-Hydroxy-pyrrolidin-2-yl)-2-oxo-propyl]-3H-pyrido[4,3-d]pyrimidin-4-one (II): Synthetic procedures follow the protocols of compound I. Compound II (di-HCl salt) is pale-yellow crystal. Yield: 85%. MP: 251° C. [α]²⁵_{D} −31.4 (c 0.5, EtOH). ¹H NMR: 9.64 (s, 1H), 9.18 (d, J=7.5 Hz, 1H), 8.72 (s, 1H), 8.14 (d, J=7.5 Hz, 1H), 5.07 (d, J=17.4 Hz, 1H), 4.90 (d, J=17.4 Hz, 1H), 4.39 (m, 1H), 4.18 (m, 1H), 3.46-3.41 (m, 2H), 2.94 (m, 1H), 2.85 (dd, J=15.1 and 6.1 Hz, 1H), 2.23 (m, 1H), 1.87 (m, 1H); ¹³C NMR: 202.6, 166.6, 163.6, 159.1, 154.5, 149.0, 147.1, 135.2, 80.3, 56.9, 55.3, 50.9, 44.3, 35.6.

3-Benzyloxy-2-[3-(5-fluoro-4-oxo-4H-quinazolin-3-yl)-2-hydroxy-propyl]-pyrrolidine-1-carboxylic acid tert-butyl ester (XVII): Synthetic procedures follow the protocols of compound XIII. Compound XVII is white solid. MP: 219° C. Yield: 78%. ¹H NMR: 8.31 (s, 1H), 8.22 (d, J=7.2 Hz, 1H), 7.81 (d, J=7.2 Hz, 1H), 7.62 (t, J=7.2 Hz, 1H), 7.34-7.28 (m, 5H), 5.23 (br, 1H), 4.48 (s, 2H), 4.30-4.21 (m, 3H), 3.89 (m, 1H), 3.63-3.55 (m, 2H), 3.39 (m, 1H), 2.24 (m, 1H), 1.88 (m, 1H), 1.55 (m, 2H), 1.45 (s, 9H); ¹³C NMR: 164.5, 163.0, 156.3, 154.5, 148.8, 147.9, 144.8, 139.0, 131.7, 128.2, 126.9, 126.4, 122.3, 80.5, 74.9, 70.3, 69.1, 66.6, 53.5, 50.2, 41.9, 38.1, 28.6.

3-Benzyloxy-2-[3-(5-fluoro-4-oxo-4H-quinazolin-3-yl)-2-oxo-propyl]-pyrrolidine-1-carboxylic acid tert-butyl ester (XVIII): Synthetic procedures follow the protocols of compound XIV. Compound XVIII is white solid. MP: 221° C. Yield: 88%. [α]²⁵_{D} +20.3 (c 0.5, EtOH). ¹H NMR: 8.29 (s, 1H), 8.21 (d, J=7.4 Hz, 1H), 7.71 (d, J=7.4 Hz, 1H), 7.63 (t, J=7.2 Hz, 1H), 7.34-7.28 (m, 5H), 5.02 (d, J=17.2 Hz, 1H), 4.86 (d, J=17.2 Hz, 1H), 4.47 (m, 2H), 4.39 (m, 1H), 4.15 (m, 1H), 3.49-3.43 (m, 2H), 2.87 (m, 1H), 2.72 (dd, J=15.2 and 5.8 Hz, 1H), 2.35 (m, 1H), 1.89 (m, 1H), 1.44 (s, 9H); ¹³C NMR: 204.1, 165.2, 160.6, 158.6, 155.9, 151.1, 149.6, 147.7, 137.6, 133.4, 128.6, 127.2, 126.9, 121.8, 79.6, 76.2, 70.5, 54.8, 53.5, 51.6, 44.9, 36.8, 28.4.

5-Fluoro-3-[3-(3-hydroxy-pyrrolidin-2-yl)-2-oxo-propyl]-3H-quinazolin-4-one (III): Synthetic procedures follow the protocols of compound I. Compound III (di-HCl salt) is pale-yellow crystals. Yield: 82%. MP: 242° C. [α]²⁵_{D} −33.8 (c 0.5, EtOH). ¹H NMR: 8.31 (s, 1H), 8.20 (d, J=7.1 Hz, 1H), 7.72 (d, J=7.2 Hz, 1H), 7.58 (t, J=7.1 Hz, 1H), 5.10 (d, J=17.4 Hz, 1H), 4.93 (d, J=17.4 Hz, 1H), 4.43 (m, 1H), 4.10 (m, 1H), 3.48-3.40 (m, 2H), 2.82 (m, 1H), 2.77 (dd, J=15.2 and 5.8 Hz, 1H), 2.28 (m, 1H), 1.93 (m, 1H); ¹³C NMR: 203.1, 164.1, 158.5, 152.7, 149.3, 147.5, 132.8, 132.2, 129.6, 80.2, 58.6, 53.2, 50.9, 43.8, 35.9.

3-Benzyloxy-2-[3-(5,6-difluoro-4-oxo-4H-quinazolin-3-yl)-2-hydroxy-propyl]-pyrrolidine-1-carboxylic acid tert-butyl ester (XIX): Synthetic procedures follow the protocols of compound XIII. Compound XIX is white solid. MP: 218° C. Yield: 83%. ¹H NMR: 8.22 (s, 1H), 7.78 (d, J=6.8 Hz, 1H), 7.69 (d, J=6.8 Hz, 1H), 7.35-7.30 (m, 5H), 5.28 (br, 1H), 4.51 (s, 2H), 4.38-4.16 (m, 3H), 3.79 (m, 1H), 3.68-3.57 (m, 2H), 3.41 (m, 1H), 2.23 (m, 1H), 1.86 (m, 1H), 1.55 (m, 2H), 1.45 (s, 9H); ¹³C NMR: 162.4, 160.2, 157.2, 152.6, 149.3, 148.3, 142.7, 138.4, 131.5, 128.6, 127.1, 126.8, 123.1, 81.1, 75.4, 70.9, 69.1, 67.3, 51.47 51.1, 40.9, 38.2, 28.5.

3-Benzyloxy-2-[3-(5,6-difluoro-4-oxo-4H-quinazolin-3-yl)-2-oxo-propyl]-pyrrolidine-1-carboxylic acid tert-butyl ester (XX): Synthetic procedures follow the protocols of compound XIV. Compound XX is white solid. MP: 211° C. Yield: 79%. [α]²⁵_{D} +19.8 (c 0.45, EtOH). ¹H NMR: 8.29 (s, 1H), 7.85 (d, J=7.1 Hz, 1H), 7.74 (d, J=7.1 Hz, 1H), 7.36-7.30 (m, 5H), 5.09 (d, J=17.1 Hz, 1H), 4.87 (d, J=17.1 Hz, 1H), 4.47 (m, 2H), 4.35 (m, 1H), 4.13 (m, 1H), 3.48-3.41 (m, 2H), 2.88 (m, 1H), 2.71 (dd, J=15.1 and 5.7 Hz, 1H), 2.34 (m, 1H), 1.79 (m, 1H), 1.44 (s, 9H); ¹³C NMR: 202.9, 164.1, 160.9, 159.2, 154.3, 150.5, 147.1, 144.8, 137.6, 132.4, 128.4, 127.4, 126.5, 120.8, 80.5, 75.7, 70.5, 55.6, 52.6, 51.7, 42.5, 34.7, 28.4.

5,6-Difluoro-3-[3-(3-hydroxy-pyrrolidin-2-yl)-2-oxo-propyl]-3H-quinazolin-4-one (IV): Synthetic procedures follow the protocols of compound I. Compound IV (di-HCl salt) is pale-yellow crystal. Yield: 91%. MP: 241° C. [α]²⁵_{D} −11.8 (c 0.5, EtOH). ¹H NMR: 8.28 (s, 1H), 7.81 (d, J=6.9 Hz, 1H), 7.72 (d, J=6.9 Hz, 1H), 5.06 (d, J=17.4 Hz, 1H), 4.92 (d, J=17.4 Hz, 1H), 4.39 (m, 1H), 4.12 (m, 1H), 3.48-3.40 (m, 2H), 2.92 (m, 1H), 2.78 (dd, J=14.9 and 5.8 Hz, 1H), 2.28 (m, 1H), 1.87 (m, 1H); ¹³C NMR: 202.8, 162.8, 161.6, 158.5, 152.1, 149.4, 147.2, 132.4, 130.5, 80.7, 58.5, 53.8, 51.4, 43.6, 37.2.

3-Benzyloxy-2-[2-hydroxy-3-(4-oxo-5-trifluoromethyl-4H-quinazolin-3-yl)-propyl]-pyrrolidine-1-carboxylic acid tert-butyl ester (XXI): Synthetic procedures follow the protocols of compound XIII. Compound XXI is white solid. MP: 224° C. Yield: 78%. ¹H NMR: 8.19 (s, 1H), 7.85 (d, J=7.2 Hz, 1H), 7.65 (d, J=7.2 Hz, 1H), 7.54 (t, J=7.2 Hz, 1H), 7.33-7.27 (m, 5H), 5.23 (br, 1H), 4.52 (s, 2H), 4.41-4.20 (m, 3H), 3.91 (m, 1H), 3.65-3.53 (m, 2H), 3.36 (m, 1H), 2.19 (m, 1H), 1.83 (m, 1H), 1.56 (m, 2H), 1.45 (s, 9H); ¹³C NMR: 162.4, 153.0, 151.1, 147.8, 146.1, 139.8, 137.1, 132.5, 130.3, 127.4, 126.9, 126.6, 122.0, 117.4, 81.7, 74.6, 71.4, 68.1, 66.5, 52.7, 50.2, 41.5, 37.5, 28.5.

3-Benzyloxy-2-[2-oxo-3-(4-oxo-5-trifluoromethyl-4H-quinazolin-3-yl)-propyl]-pyrrolidine-1-carboxylic acid tert-butyl ester (XXII): Synthetic procedures follow the protocols of compound XIV. Compound XXII is white solid. MP: 218° C. Yield: 82%. [α]²⁵_{D} −27.7 (c 0.5, EtOH). ¹H NMR: 8.22 (s, 1H), 7.89 (d, J=7.1 Hz, 1H), 7.71 (d, J=7.1 Hz, 1H), 7.57 (t, J=7.2 Hz, 1H), 7.35-7.27 (m, 5H), 5.09 (d, J=17.3 Hz, 1H), 4.87 (d, J=17.3 Hz, 1H), 4.48 (m, 2H), 4.39 (m, 1H), 4.12 (m, 1H), 3.48-3.43 (m, 2H), 2.88 (m, 1H), 2.77 (dd, J=15.1 and 5.7 Hz, 1H), 2.42 (m, 1H), 1.89 (m, 1H), 1.44 (s, 9H); ¹³C NMR: 203.4, 162.1, 156.3, 150.1, 147.8, 146.3, 140.7, 135.9, 132.1, 130.4, 128.2, 126.9, 126.4, 122.9, 116.3, 79.1, 74.6, 70.6, 53.4, 52.6, 52.1, 43.7, 36.5, 28.4.

3-[3-(3-Hydroxy-pyrrolidin-2-yl)-2-oxo-propyl]-5-trifluoromethyl-3H-quinazolin-4-one (V): Synthetic procedures follow the protocols of compound I. Compound V (di-HCl salt) is pale-yellow crystals. Yield: 84%. MP: 262° C. [α]²⁵_{D} +27.5 (c 0.5, EtOH). ¹H NMR: 8.24 (s, 1H), 7.88 (d, J=7.2 Hz, 1H), 7.65 (d, J=7.2 Hz, 1H), 7.52 (t, J=7.2 Hz, 1H), 5.06 (d, J=16.8 Hz, 1H), 4.84 (d, J=16.8 Hz, 1H), 4.40 (m, 1H), 4.19 (m, 1H), 3.47-3.41 (m, 2H), 2.85 (m, 1H), 2.78 (dd, J=15.3 and 6.2 Hz, 1H), 2.27 (m, 1H), 1.93 (m, 1H); ¹³C NMR: 202.8, 162.7, 158.2, 150.5, 148.3, 141.4, 133.3, 132.7, 131.7, 116.8, 80.5, 57.6, 53.6, 50.5, 42.4, 36.5.

It is understood that while a compound of the general structural formulas herein may exhibit the phenomenon of tautomerism, the structural formulas within this specification expressly depict only one of the possible tautomeric forms. It is therefore to be understood that the structural formulas herein are intended to represent any tautomeric form of the depicted compound and is not to be limited merely to a specific compound form depicted by the structural formulas.

It is also understood that the structural formulas are intended to represent any configurational form of the depicted compound and is not to be limited merely to a specific compound form depicted by the structural formulas.

Some of the compounds of the present invention may exist as single stereoisomers (i.e., essentially free of other stereoisomers), racemates, or mixtures of enantiomers, diastereomers, or both. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically, pure), and an optically pure compound having more than one chiral center is one that is both diastereomerically pure and enantiomerically pure. Preferably, if the compounds of the present invention are made synthetically, they are used in a form that is at least 90% optically pure, that is, a form that comprises at least 90% of a single isomer (80% enantiomeric excess (e.e.) or diastereomeric excess (d.e.), more preferably at least 95% (90% e.e. or d.e.), even more preferably at least 97.5% (95% e.e. or d.e.), and most preferably at least 99% (98% e.e. or d.e.).

Additionally, the compounds of the invention include pharmaceutically acceptable salts, multimeric forms, active metabolites, precursors and salts of such metabolites of the compounds of the present invention.

The compound of the present invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyrvic acid, oxalic acid, glycolic acid, salicylic acid, a pyranosidyl acid, such as glucuronic acid or galacturonic acid, an .alpha.-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

The compound of the present invention are solids, it is understood by those skilled in the art that the compound of the present invention and salts may exist in different crystal or polymorphic forms, all of which are intended to be within the scope of the present invention and specified structural formulas.

The compounds of the present invention in accordance with the present invention are useful in the treatment malaria and diseases and disorders associated with malaria or a Plasmodium parasite.

Antimalarial Activity

The blood schizonticidal activity of synthesized pyridopyrimidinone compounds (Table I) was determined as described herein. Prophylactic efficacy of synthesized pyridopyrimidinone compounds was determined as described herein.

TABLE I
Antimalarial Activity of Pyridopyrimidinone Compounds: Blood
schizontocidal activity against P. berghei in mice:
Mice surviving 60 days/Mice infected and treated.

| | Oral Dose, mg/kg, day; (Total dose, mg/kg) | | | | |
|---|---|---|---|---|---|
| Compound # | 0.25 (0.75) | 1.0 (3.0) | 4.0 (12) | 16 (48) | 64 (192) |
| Chloroquine | 0/7 | 0/7 | 2/7 | 4/7 | 0/7 |
| Febrifugine | 1/7 | 2/7 | 5/7 | 0/7 | 0/7 |
| I | 7/7 | 7/7 | 7/7 | 7/7 | 7/7 |
| II | 7/7 | 7/7 | 7/7 | 7/7 | 7/7 |
| III | 5/7 | 6/7 | 7/7 | 7/7 | 7/7 |
| IV | 6/7 | 7/7 | 7/7 | 7/7 | 7/7 |
| V | 7/7 | 7/7 | 7/7 | 7/7 | 7/7 |
| Control | 0/7 | | | | |

Compounds I-V are very active and much less toxic (no mice showed toxicity at the highest dose tested). These compounds had also shown excellent activity by the subcutaneous route of administration [J. Med. Chem., 25, 1094(1982) M. P. LaMontagne et al.] (See Table 2).

TABLE 2
Suppressive Antimalarial Activity of Pyridopyrimidinone Compounds:
Blood schizontocidal activity against P. berghei in mice
(J. Med. Chem., 2 5, 1094(1982) M. P. LaMontagne et al.).
Subcutaneously as a single dose.
Mice surviving 60 days/Mice infected and treated

| | Dose, mg/kg | | | | | | | |
|---|---|---|---|---|---|---|---|---|
| Compounds | 1 | 2 | 4 | 8 | 16 | 32 | 64 | 128 |
| I | 4/5 | 5/5 | 5/5 | 5/5 | 5/5 | 5/5 | 5/5 | 5/5 |
| II | 4/5 | 5/5 | 5/5 | 5/5 | 5/5 | 5/5 | 5/5 | 5/5 |
| III | 4/5 | 5/5 | 5/5 | 5/5 | 5/5 | 5/5 | 5/5 | 5/5 |
| IV | 4/5 | 5/5 | 5/5 | 5/5 | 5/5 | 5/5 | 5/5 | 5/5 |
| V | 4/5 | 5/5 | 5/5 | 5/5 | 5/5 | 5/5 | 5/5 | 5/5 |
| Control | 0/5 | | | | | | | |

Mice were treated with a single dose of the compound administered subcutaneously 72 h. after infection. Number of cures is the number of mice surviving, out of five, 60 days postinfection.

Compounds were tested for oral prophylactic activity. Mice treated with a single dose of 0.5 mg/kg of compound I within the period of 2 days preinfection through 2 days post infection were completely protected from malaria. Data were summarized in Table 3 and Table 4.

TABLE 3
Prophylactic Anti-Malarial Activity of Compound I.
Mice infected and treated/Mice surviving day 60

dose, mg/kg

| Day of Treatment | 0.5 | 2 | 8 | 32 |
|---|---|---|---|---|
| −3 | 0/5 | 5/5 | 5/5 | 5/5 |
| −2 | 5/5 | 5/5 | 5/5 | 5/5 |
| −1 | 5/5 | 5/5 | 5/5 | 5/5 |
| 1 | 5/5 | 5/5 | 5/5 | 5/5 |
| 2 | 5/5 | 5/5 | 5/5 | 5/5 |

TABLE 4
Prophylactic Anti-Malarial Activity of Compounds II–V (single
dose of 0.5 mg/kg).

| | Day of | Mice surviving day 60/Mice |
|---|---|---|
| Compounds | Treatment | infected and treated |
| II | −2 | 5/5 |
| III | −2 | 5/5 |
| IV | −2 | 5/5 |
| V | −2 | 5/5 |
| II | −1 | 5/5 |
| III | −1 | 5/5 |
| IV | −1 | 5/5 |
| V | −1 | 5/5 |
| II | 1 | 5/5 |
| III | 1 | 5/5 |
| IV | 1 | 5/5 |
| V | 1 | 5/5 |
| II | 2 | 5/5 |
| III | 2 | 5/5 |
| IV | 2 | 5/5 |
| V | 2 | 5/5 |
| Controls | | 0/5 |

The present invention, hence, relates to the finding that some pyridopyrimidinone compounds have significantly greater activity and/or less toxicity than the original natural product febrifugine in the treatment of malaria. The present invention relates to new pyridopyrimidinone compounds compositions and also to the use of these compositions as pharmaceuticals when combined with an acceptable pharmaceutical carrier in the treatment of malaria. The present compositions can also be used as prophylactics for the prevention of malaria.

Administration of the compounds of the invention may be parenteral, oral, intravenous, intramuscular, subcutaneous, intrapleural, intrathecal, intraperitoneal, aerosol or transdermal administration to achieve the desirable antimalarial effect. These drugs may be administered as the free base form or in the form of a pharmaceutically acceptable acid addition salt wherein the acid addition salt may be either organic or inorganic in nature. Suitable inorganic acids for salt formation include but are not restricted to: phosphoric acid, hydrochloric acid or sulfuric acid. Suitable organic acids for the formation of salts may include but are not restricted to: succinic acid, citric acid, fumaric acid or isothionic acid. When administered orally, the compounds of the invention may be in the form of tablets (single or multilayer, coated or uncoated) capsules or dragees. These oral formulations may be admixed with a solid excipient such as lactose, sucrose, starch, microcrystalline cellulose, magnesium sterate, or talc. When parenteral administration may be indicated, an aqueous solution or an oleaginous formulation of the agent may be employed. Aqueous solutions can be prepared in water or physiological saline, either with or without buffers. Oleaginous formulation may be made in natural oils such as peanut oil or olive oil, for example. The actual dosage amount administered can be determined by physical and physiological factors such as body weight, severity of condition, and idiopathy of the subject.

Biological Testing Procedures in Animals

Blood Schizontocidal Test

Drugs were mixed in 0.5% hydroxycellulose 0.1% Tween 80 and administered orally b.i.d. on days 3, 4 and 5 postinfection. CD-1 male or female mice, 5 weeks of age, were infected with 5.times.10.sup.4 parasitized erythrocytes of Plasmodium berghei KBG-173 mm strain. Blood films were taken on day +6 and weekly thereafter until day +60. Parasitemias were calculated and SD90 value (dose suppressing 90% of the parasites in treated groups compared with the infected non-treated controls) on day +6 postinfection. Mortality data was tabulated for 60 days at which time all mice surviving that were blood film negative were considered cured.

Compounds were tested at three dose levels, 4, 1, and 0.25 mg/kg body weight per day. The activity of these compounds were compared with the untreated control. In untreated controls, death occurs within 8-9 days. Compounds which are effective against Plasmodium berghei infection increase the mean survival time of the infected animals when compared with the untreated controls. Mice that survive after thirty days and are free of parasites in blood are considered cured.

Efficacy of the drug is determined by the number of cures at the end of a 30 day period and the increase in mean survival time over the control (.DELTA.MST). The effect of the test drugs also could be determined by the reduction of the parasitemia (percentage of the red blood cells detected with the parasites) over the untreated control on day 6, one day after the treatment is completed. Both these methods yield virtually identical results. If the dose of test compounds are inadequate, after initial clearance, residual parasites will multiply and relapses will occur within thirty days.

Prophylactic Test

Drugs were mixed in 0.5% hydroxycellulose 0.1% Tween 80 and administered orally b.i.d. either on day 5, 4, 3, 2, or 1 prior to the infection or 1 or 2 days postinfection. CD-I male or female mice, 5 weeks of age, were infected with 5.times.10.sup.4 parasitized erythrocytes of Plasmodium berghei KBG-173 mm strain. Blood films were taken on day +6 and weekly thereafter until day+30. Mortality data was tabulated for 30 days at which time all mice surviving that were blood film negative were considered cured.

Unless defined otherwise, all technical and scientific terms used herein have the same meaning as commonly understood by one of ordinary skill in the art to which this invention belongs. Although any methods and materials similar or equivalent to those described can be used in the practice of testing of the present invention, the preferred methods and materials are now described. All publications and patent documents referenced in this application are incorporated herein by reference in their entirety as though each and every publication and patent document was specifically incorporated herein by reference in its entirety.

It is understood that the examples and embodiments described herein are for illustrative purposes only and the various modifications or changes in light thereof will be suggested to persons skilled in the art and are to be included within the spirit and purview of this application and scope of the appended claims.

What is claimed is:

1. A composition for the prevention or treatment of malaria, the composition consisting of one of the following compounds: (I) 3-[3-(3-Hydroxy-pyrrolidin-2-yl)-2-oxo-propyl]-3H-pyrido[3,2-d]pyrimidin-4-one; (II) 3-[3-(3-Hydroxy-pyrrolidin-2-yl)-2-oxo-propyl]-3H-pyrido[4,3-d]pyrimidin-4-one; (III) 5-Fluoro-3-[3-(3-hydroxy-pyrrolidin-2-yl)-2-oxo-propyl]-3H-quinazolin-4-one; (IV) 5,6-Difluoro-3-[3-(3-hydroxy-pyrrolidin-2-yl)-2-oxo-propyl]-3H-quinazolin-4-one; (V) 3-[3-(3-Hydroxy-pyrrolidin-2-yl)-2-oxo-propyl]-5-trifluoromethyl-3H-quinazolin-4-one.
